(19) United States
(12) Patent Application Publication
(10) Pub. No.: US 2020/0360426 A1
(43) Pub. Date: Nov. 19, 2020

(54) PROTOCOL AND COMPOSITION FOR SUPPRESSION OF ALLERGIC RESPONSES

(71) Applicant: ISAAC ELIAZ, SANTA ROSA, CA (US)

(72) Inventor: ISAAC ELIAZ, SANTA ROSA, CA (US)

(21) Appl. No.: 16/524,123

(22) Filed: Jul. 28, 2019

Related U.S. Application Data
(60) Provisional application No. 62/849,084, filed on May 16, 2019.

(51) Int. Cl. A61K 33/06; A61K 9/08; A61K 45/06; A61J 1/10

(52) U.S. Cl. CPC A61K 33/06; A61K 9/08; A61J 2205/30; A61J 1/10; A61K 45/06

(57) ABSTRACT

A protocol for limiting the severity of allergic reactions, or preventing them entirely, is provided. The central feature of the protocol is the delivery, over a period of no more than an ninety minutes, of an amount of magnesium ranging from 50-500 mg magnesium, measured as elemental magnesium. A wide variety of salts and chelates of magnesium may be used. The magnesium is deliver via IV, at a relatively high rate, of no more than ninety and preferably about thirty minutes. IV bags, prepared for use in this invention, reflecting the instructions for use set forth above, are claimed as well.

### CLAIM TO BENEFIT

Applicant claims benefit of the filing date of U.S. Provisional Patent Application Ser. No. 62/849,084 filed May 16, 2019 for purposes of priority.

### BACKGROUND OF THE INVENTION

### Field of the Invention

The present invention relates to a protocol and composition effective to suppress allergic reactions in mammalian patients, particularly humans. Patients encounter allergic reactions which can range from mild to severe and even lethal allergic responses following exposure to a wide variety of allergens—everything from medication to naturally existing products to newly encountered foods and beyond. The new protocol suppresses these reactions and relies on a preparation for intravenous infusion (IV) of effective agents so as to administer the protocol rapidly and effectively.

### SUMMARY OF THE INVENTION

Most individuals, humans and comfort and commercial mammals, experience allergic responses. While the source or cause of the allergic response can vary from drugs administered to the patient, sinusoidal irritation caused by airborne environmental toxins like pollen, unfamiliar food that is not ingested, foreign proteins such as those resulting from insect bites and stings, artificial complexes like latex and the like, and a whole host of other triggering agents. The degree or severity of the reaction is often difficult to immediately diagnose or judge, but can range from conventional itching or sneezing all the way up to anaphylactic shock in which blood pressure drops and the situation can become lethal if not counteracted.

This application is related to, and claims benefit of the filing date of, U.S. Provisional Patent Application Ser. No. 62/849,084 filed May 16, 2019. That application is directed to a very narrow range of anaphylactoid treatment protocols, addressing the consequence of using dextran sulfate apheresis columns for LDL cholesterol removal, and for removal of LP(a) and CRP, as well as for the removal of other compounds from the plasma. The apheresis process may include separation of blood components, followed by return of most of those components to the patient by retransfusing the whole blood back to the patient. In most apheresis devices and treatments, blood cells are separated from plasma initially, and the plasma is subsequently treated to at least remove a blood component before return to the individual. Blood cells, which typically entrain platelets and related blood components, may also be treated (or collected for specific purposes such as to provide donor material). By removing the blood and plasma from the body, treatment of a wide variety of conditions and disorders becomes simplified, without having to deal, at least initially, with natural responses and side effects, such as cytokine-triggered inflammation responses present when the treatment is effected by administration within the body.

In existing commercial apheresis methods, the blood of the patient/donor is run past or through a column, to remove toxic substances from the blood and restore homeostasis. In one embodiment, this column comprises Dextran Sulfate. One example of such a column is the Liposorber™ apheresis column distributed by Kaneka Pharma America, LLC. The information available from the FDA on this column and system provides “the LIPOSORBER LA-15 System is a blood processing system that collects blood from the patient's body and removes certain substances such as low density lipoprotein cholesterol (LDL-C, or “bad cholesterol”) and other harmful substances from the patient's blood. The system also helps slow the progression of an aggressive kidney disease called Focal segmental glomerulosclerosis (FSGS).”

The information provided specifically informs the reader/user the Liposorber 15-LA System® using the Liposorber column traps and removes LDL-cholesterol in the blood. By removing the bad cholesterol, Kaneka indicates, the system may reduce the likelihood of developing serious disease of the heart and blood vessels. The system may also trap and remove other harmful substances contributing to the progression of the kidney disease. The system also helps slow the progression of FSGS in adult and pediatric patients. The information available from the FDA also specifically observes that a large number of allergic reactions have been experienced by those using the Liposorber 15-LA® column. These allergic reactions have become more widespread, and are apparently more severe in apheresis patients taking angiotensin-converting-enzyme (ACE)-inhibitors which are prescribed, distributed and administered to reduce blood pressure.

In the Liposorber 15-LA® system, the apheresis column used comprises dextran sulfate. Some of dextran sulfate can leech into the blood. In other patients, the reaction develops in the apheresis column filter itself. A typical reaction is flushing, heat, vaso-vagal like reaction, hypotension, bradycardia. It is mediated by Bradikinin, causing angio-reactivity, angioedema like reaction. Previously, a number of patients who cannot tolerate the dextran sulfate Liposorber column reaction had used an alternate apheresis system offered as the BBraun HELP System™. That system is no longer available, leaving these patients suffering from hypercholesterolemia with little or no alternative for treatment.

Those of skill in the art, which typically includes experienced medical practitioners, are aware of the bradykinin-mediate allergic reactions that can be experienced with the use of a dextran sulfate apheresis column. Bambauer R, et al, LDL-apheresis: Technical and Clinical aspects. Scientific World Journal 2012:314283, reports the effective use of dextran sulfate columns but also noting that drugs that inhibit angiotensin converting enzyme (ACE inhibitors) can exacerbate the clinical effect of (this amount of-?) bradykinin to the point of serious adverse effect including anaphylaxis. Report also indicate that Angitoensin (II) Receptor Blocker medications (ARBs) which are also commonly administered to patients suffering from high blood pressure, kidney and liver failure also cause or aggravate the allergic reaction.

The inventor's work in treating and suppressing the severe reaction to apheresis that has been encountered using dextran sulfate, with no alternative treatment available, has led to a recognition that not just anaphylactic shock allergic reactions induced while using dextran sulfate apheresis can be suppressed or avoided, but that the same protocol, broadened and expanded, can be used to address a wide variety of allergic conditions, including but not limited to, anaphylactic and anaphylactoid shock as well as other allergic reactions.

### SUMMARY OF THE INVENTION

Magnesium, available in a variety of forms including MgSO₄, magnesium hydroxide (Mg(OH)₂], magnesium chloride, (MgCl₂), magnesium citrate (C₆H₆O₂Mg) has long been used in treating specific mammalian conditions. Magnesium acetate for example is used as a supplement and been shown effective in treating morphine dependency, Rabbani et al, Magnesium Research 2012; 15(1):40-8, while MgO may be effective in the treatment of calcium oxalate nephrolithiasis. Khan et al, Journal of Urology, 149, 2; 1998, 412-416. MgCl has been used in the treatment of heart arrhythmias following coronary bypass surgery, England et al, JAMA, 1992; 268 z(17):3395-3402, while Kim et al, PLOS ONE 12(3):e0173026, https//doi.org/10.1371 discuss the utility of using magnesium sulfate in addressing regional pain following surgery like mastectomies. Most frequently, magnesium sulfate and similar magnesium salts have found applicability in the treatment of ventricular arrhythmias. Iseri L T, Magnesium, 1986; 5:111-12.

Much is known about magnesium and its common salts—magnesium acts as a cofactor with a wide variety of enzymes. It also serves a variety of membrane functions including cell adhesion, transmembrane electrolyte flux control, neurotransmitter release and the like. In still other modes, magnesium serves as a structural agent in proteins, nucleic acids, mitochondria and more. Thus, magnesium is well known as a constituent agent and actor in many different aspects of the mammalian patient responses, and is well tolerated. The inventor has discovered that magnesium, most commonly administered as magnesium sulfate but considered herein as elemental magnesium or magnesium IV, is effective in preventing, suppressing or quieting allergic reactions of all kinds. The invention embraces preventing allergic reactions by administering magnesium in anticipation of possible events giving rise to potential allergic reactions, such as apheresis or transfusion. Importantly, the invention also is directed to addressing and ameliorating, reducing and possibly suppressing allergic reactions once they are initiated.

The protocol set forth in previously mentioned U.S. Provisional Patent Application Ser. No. 62/849,084 filed May 16, 2019, incorporated herein-by-reference in its entirety, illustrates this principle in a narrow framework. That application proposes to treat allergic responses to dextran sulfate encountered in apheresis by administration of magnesium, together with a histamine blocker, such as Zyrtec®. It is also important to minimize the vaso-vagal reflex and associated nausea and GERD. To this end an antacid, such as Pepcid® or Pepcid AC® (famatodine) is administered. Both the histamine blocker and the antacid are given prior to apheresis. To provide for maximum effectiveness, the antacid and the histamine blocker are administered 45 minutes to an hour before apheresis. The magnesium (magnesium sulfate) is infused just before apheresis with the dextran sulfate column begins.

The inventor has now discovered that even less extreme allergic reactions, or extreme reactions induced by other causes and conditions, may be treated through the same fashion, by IV administration of magnesium, before allergenic treatment as a preventative measure, or as soon after development of an allergic reaction is detected or projected Thus, where a mammalian patient is identified as having ingested or received via other routes a composition or compound known to cause allergic reactionsin that patientone need not wait until the allergic reaction is manifested to initiate treatment with magnesium. In addition, in advance of events which either historically or predictably generate allergic reactions in a patient, such as blood transfusions, plasma exchange, and similar practices, magnesium administered in advance of the treatment may prevent an allergic reaction from developing. An early administration of magnesium can effectively prevent or minimize an allergic reaction that might be expected on the basis of historical incidents. The administration of magnesium can be a stand-alone treatment, or be combined with other agents, as is the case with the dextran sulfate apheresis, where an antacid and histamine blocker are also administered. The administration of magnesium IV called for by this invention can be combined with a variety of aids intended to further reduce or control allergic reactions. Thus, the magnesium may be administered together with an immune suppressant or with corticosteroids. The co-administered agents may be either long acting or relatively quick acting. While the magnesium of the invention is administered IV, the co-acting agents may be administered orally or via inhalation. They may also be administered IV, on their own or/and together with the magnesium. The invention is also advantageously combined with administration of sympathetic nervous system antagonists such as B-blockers or any of a variety of the co-acting agents discussed below. These include agents that block activation of all types of β-adrenergic receptors as well as others that are selective for one of the three known types of beta receptors, designated β1, β2 and β3 receptors.

In addition to the administration IV of magnesium, various other factors are advantageously taken into account in designing a treatment specific for each patient. While the invention provides a rapid response for allergic reactions that are unexpected or occur without warning, in a variety of environments, the patient or care giver may be aware that allergic reactions are anticipated or possible, and pretreatment is then advantageously given. The specific character of pretreatment depends on a number of factors. These include:

1. Condition and treatment addressed—what is the agent that is likely to be the cause of the allergic reaction to the treatment or procedure to be employed. Examples of this factor are discussed above, e.g., Liposorber LDL apheresis, blood transfusion, etc.

2. Patient's history and co morbidities—Often a patient that has a particular or uncommon history needs special care and consideration when an action that may provoke an allergic reaction is being contemplated. History may be considered to include specific genetic dispositions that can be tested or detected through a wide variety of gentic testing protocols. This may also include various SNP expression such as MTHFR expression of C677T and A1298C.

3. Other treatments that are necessary for the care of the patient—Although it may seem straightforward in an independent consideration, often time patients are undergoing various treatments that may be unrelated to the treatment that might induce an allergic response. Knowing the full medical history and treatment of the patient is invaluable in predicting when and how to administer agents in addition to magnesium, in advance, to suppress or delimit.

4. Status of Phase I and phase II detoxification in the liver. Given the information identified above, different co factors and adjuvant treatments can be utilized, prior to the administration of magnesium, during and after. The companion treatments may be given orally, in an injection form (subcutaneous, intramuscular or intravenous) If administered IV, the companion or support treatments can be mixed in the same bag with Mg, or given separately. Examples of these additional treatments include the various B vitamins such as thiamine (B1), riboflavin, niacine and nacine amide (nicotinamide), panthotenic acid, B-6, B-12. Glycine, Methionine, N-Acetyl cystein, MSM 9 (methyl-sulfonyl-methane). glutathione and guthatione synthetase cofactors, reduced glutathioine, alpha lipoic acid may also be used to support or extend treatment of patients in need of the magnesium administration of this invention (for instance, diabetic patients may require supplements of alpha-lipoic acid). A wide variety of mineral and trace elements such as selenium, zinc, copper, chromium, and other trace elements, as well as botanical supplements such as milk thistle may be used in connection with the administration of magnesium contemplated by this invention etc. Homeotoxicology preparations such as lymphomyosot, hepar compositum, ubichonone, solidago, traumeel, and similar preparations may often be used by patients encountering treatments likely to trigger allergic reactions, including various apheresis, dialysis, transfusions and blood product additions and thus effectively coadministered with this invention. Other vitamins, especially vitamins, A, C, E, and D may be co-adminstered with the magnesium agent of this invention. (Treatments involving lipid exchange where lipid soluble toxins are removed using phosphatydil choline (PC), followed by glutathione, can be co-administered IV with, or as part of the magnesium IV protocol. Similar supportive treatments include folate supplementation (5-MTHF) alone or with PC and glutathione protocol can be used in patients who may receive elements likely to induce an allergic reaction and may be combined with this invention. Other co-administration protocols include administration of charcoal and similar binders to bind toxins, modified citrus pectin, regular pectins, alginates, sea weeds and other lectin binders. All of these supportive treatments may be combined with the administration of magnesium called for by this invention where it is foreseeable that an allergic reaction may be raised in a patient that should be avoided.

### DETAILED DESCRIPTION OF THE INVENTION

This invention finds application in every environment where allergic reactions are encountered. There are a large number of conditions and situations that may lend themselves to this protocol. This includes things like transfusions of blood products, packed red blood cells, whole blood, platelets and the like. As noted, and discussed in more detail below, this invention lends itself to the treatment of allergic reactions developed in response to apheresis of all kinds. In many kinds of plasma exchange, citrates are used as anticoagulants, which may independently trigger allergic reactions. Stem cell collections, via apheresis and other methods, can often give rise to allergic reactions. Mammalian patients exhibit allergic reactions to a wide range of IV drug therapies, including taxane, platinum chemotherapy, immunotherapy, herceptin, etc. Similar therapies, like tyrosine kinase inhibitors, PD-1 and PDL-1 inhibitors often produce or aggravate allergic reactions. Tumor infiltrating lymphocyte treatment (TIL) may cause an allergic reaction or/and a cytokine storm as well. By the same token, dialysis in general may induce allergic reactions to the dialyzer, and IV replacement therapy, (albumin, amino acid, TPN, blood products, etc) may generate allergic reactions. Wherever non-naturally generated products are introduced to the blood or circulation of a mammalian patient, allergic reactions may occur. Frustratingly, individuals vary by a great deal—and no generic prediction or expectancy of allergic reactions can be arrived at.

As a response to allergic reactions to any of these conditions or sources, and many others, the inventor has developed a protocol and product that alleviates and suppresses the severity of that reaction, either providing complete relief to the patient, or deferring for a substantial period of time the severity of the allergic reaction until the patient's specific condition can be treated by specific methods. In the protocol of this invention, magnesium is administered IV to the patient over a relatively short period of time. In the ideal situation, magnesium is administered before the beginning of the procedure of concern. It can also be repeated during the procedure, and after the procedure as well. By acting prior to the initial cause of the allergic response anticipated, it may be suppressed to some degree or even avoided. While virtually any of the magnesium salts discussed above can be used, magnesium sulfate is common, and serves as the basis for the discussion herein, based on elemental magnesium.

The IV drip is introduced to the patient rapidly. Again, as soon as the allergic reaction itself is identified, or an expectancy of the onset of an allergic reaction due to prior behavior is identified, the magnesium may be administered. It can and should be administered to all patients undergoing these procedures prior to starting the procedure in order to reduce the allergic side effects and improve tolerance. While an IV induction of up to 60 minutes may be used, preferred drip times are 20-45 minutes, with 25-30 minutes being highly preferred. Considering the agent as elemental magnesium, independent of the salt or chelate, administration of 50-750 mg of elemental magnesium, preferably 100-175 mg, and most preferably 125-175 mg elemental magnesium is administered pursuant to this invention. To that end, IV drip preparations or bottles of magnesium solution are prepared with varied amounts of magnesium based on elemental Mg, and stored, so as to provide for a very rapid response. An IV drip can be up and running within a very few minutes of detection of the allergic response or expectancy of that response, and if not suppressed, reduce the severity of the allergic reaction substantially. Such pre made bags can include specific cofactors as detailed above prepared for a specific condition or patient.

### Specific Example

As noted above, a very special or select aspect of this invention is disclosed in U.S. Provisional Patent Application Ser. No. 62/849,084 filed May 16, 2019. That detailed discussion is confined to addressing the very sharp allergic reactions to dextran sulfate experienced by a large percentage of the public receiving lipidemia apheresis (as high as 12% of those receiving treatment. In that specific embodiment, both an antihistamine and an antacid may be co-administered with the magnesium but are not essential to achieving the goals of the invention as claimed below.

A fair amount of information has been developed on the complicated interrelationship between medications like the ACE inhibitors and ARBs, particularly for patients at risk for kidney disease. Outside of the claimed invention, the art does not provide a specific solution for dextran sulfate allergic reactions, but it does discuss the various agents and mediators involved in the involved pathway. Cunha et al, Magnesium and Vascular Changes in Hypertension, Intl. Journal of Hypertension, 2012. The investigators report the mineral magnesium, often involved as an intracellular cation, is involved in several important biochemical reactions. Magnesium has antiarrhythmic effect and can influence blood pressure levels by modulating vascular tone. Changes in extracellular magnesium content have been seen to modify the production and release of nitric oxide (NO), resulting in the alteration of arterial smooth muscle tone by affecting calcium concentrations. Magnesium also participates in glucose metabolism and insulin homeostasis. For these reasons, it has been suggested that magnesium deficiency or changes in its metabolism are related to the pathophysiology of hypertension, atherosclerosis, insulin resistance, and diabetes.

Based on experience and knowledge of the effects of apheresis, the inventor has found a pre-apheretic protocol that permits even those sensitive to dextran sulfate, and likely to exhibit an anaphylactic reaction to such apheresis, to undergo this frequently life-saving reaction. The protocol begins with the oral administration of an antacid and a histamine blocker. The specific agent employed, provided it supplies the necessary suppression, does not matter. As potential antacids, one may select from a variety of commercially available products, including Alka-Seltzer®, Milk of Magnesia®, Alternagel, Amphojel, Gaviscon®, Gelusil®, Maalox®, Mylanta®, Rolaids,® Pepto-Bismol®, and Tum®s. The histamine blocker can be selected from a number of commercial alternatives. These include, specifically, H-1 blockers, such as loratadine, fexofenadine and azelastine. Commercially available H-1 blockers include products such as Claritin®, Allegra®, Zyrtec® and Astelin®.

The administration of an antacid and a histamine blocker, alone, will not effectively suppress the allergic reaction to dextran sulfate. Centrally important and essential is the administration of magnesium, preferably magnesium IV, available as magnesium sulfate. Administration of magnesium sulfate, shortly in advance of apheresis, serves to relax the vascular system, and shift the patient or a sympathetic response to a parasympathetic vascular relaxation response. Magnesium realizes and calms the sympathetic FFF response, the survival instinct that is so prevalent in angioedema. As such it calms or eliminates the allergic reaction to dextran sulfate. Infusing at least 1.5 grams magnesium sulfate and/or chelate, preferably magnesium sulfate, is a central step in this improved protocol. While it can be infused over a longer period of more than one hour, a preferred protocol infuses it very rapidly. It can be infused in as little as 15 minutes, but best results have been obtained by infusing it over 25-30 minutes. A drip in 100 ml-250 ml normal physiologic saline solution (Ns) or lactated Ringers solution (LR) has been employed. Other carriers, such as dextrose in water (5%) (D5W) or sterile water at smaller volumes, or similar physiologically compatible carriers may be used. A faster drip of not more than 30 minutes is preferable, and may be essential to intervene in and reduce ongoing severe reactions. Magnesium sulfate may be obtained as the single salt, commonly referred to as Epsom Salts. It is also available as the hydrate, the heptahydrate and in solution. It is available for IV use as 50% concentration, meaning 500 mg per 1 ml. As noted, while the protocol advantageously includes the use of an antihistamine and an ant-acid, it is also effective with the use of Magnesium IV alone.

The administration of magnesium, a histamine (H-1) blocker and antacid in advance of the apheresis (up to 60 minutes in advance) is sufficient to ensure most allergic reactions to dextran sulfate during apheresis are suppressed. As has been discussed above, ACE inhibitors should be discontinued well in advance of the apheresis procedure, perhaps as much as a week in advance. ARB's appear to be less problematic, but discontinuation of ARBs is also advisable, particularly in sensitive patients. During the apheresis oxidized lipids are removed. When the apheresis is done, dextran sulfate exposure can still cause a reaction. For susceptible patients another 1-1.5 grams of magnesium Sulfate or elemental magnesium equivalent in 100 ml NS may be given at the end of apheresis as well. Apheresis patients also get oxygen via canula, 2-4 liters a minute through the procedure. The administration or giving of oxygen helps to prevent the hypoxic acidotic, and a survival, sympathetic response.

The protocol described above was administered to apheresis patients. In each case, the apheresis column employed was a dextran sulfate column from Kaneka Pharma America, LLC. In a total of at least 60 apheresis treatments, treating at least thirty different patients, no issues and little or no allergic reactions were observed. In each case, the full protocol observed above, including cessation of ACE inhibitors at least a week prior to apheresis, administration of an antacid and a H-1 histamine blocker, was followed. In the preferred embodiment, no more than an hour before apheresis, magnesium sulfate is administered as described above. At the end of apheresis, for sensitive patients, it was accompanied by 1-1.5 grams of magnesium sulfate or equivalent in 100 ml Ns. Oxygen, via canula, was administered throughout the apheresis for many of the patients. For seven patients who previously had an anaphylactoid shock reaction with the use of Liposorber discussed above, even with the concomitant use of IV steroids, the use of magnesium sulfate without any supportive or additional treatments allowed all 7 patients to tolerate the treatment.

Given the absence from the marketplace in the United States of a practical apheresis alternative that doesn't include dextran sulfate, and the lifesaving nature of lipoprotein apheresis, a solution that permits the very sizable minority of patients with a bradykinin mediated allergic reaction to dextran sulfate is of significant importance. None of the protocol steps described herein is harmful or dangerous in any way to potential apheresis candidates. Accordingly, the protocol described herein is beneficially provided to all patients about to undergo an apheresis treatment employing the Liposorber column provided by Kaneka, or any other dextran sulfate-based column.

The same considerations expressed above in the context of this invention are generally applicable. In some situations, co-administration of other agents implicated by the condition addressed or standard medications may be called for. The core of the invention, however, is the rapid delivery of magnesium IV, preferably magnesium sulfate, although other magnesium salts and chelates, such as hydroxide, acetate, magnesium hydroxide, magnesium chloride, magnesium citrate, MgO and MgCl may be used. This may be combined, where appropriate, with administration of corticosteroids, Imuran (Azathioprine), B-blockers such as Deralin (propranolol hydrochloride), mast cell stabilizers like Cromolyn (asthma suppressant), anti-emetics like Zofran, etc.

This application is directed to those of general skill in this art. This invention does not require the intervention of a medical doctor. While the skills and experience of a nurse may be of value, patients well familiar with the details and requirements of various treatments like apheresis or dialysis may well be able to administer it themselves, given the time and amount restrictions discussed above, which will be reflected on the label firmly affixed to the IV bag or container. The same individuals will recognize that by administration or administering the magnesium, Applicant is referring to the provision or giving of the magnesium as described, in this case via intravenous infusion. IV administration is important in this particular invention to deliver the magnesium, and forestall or suppress an allergic reactions to the greatest degree possible. Administration can occur in an office, at a facility or at home. By the same token, recognizing or identifying a patient as one who may suffer an allergic reaction requiring or supporting intervention may be assisted by a medical practitioner, but need not be if experience with allergic reactions or a particular protocol is available to the patient. Identifying a patient who is suffering from an allergic reaction may further be based on the experience and observations of one familiar with these situations.

What is claimed is:

1. A method of treating a mammalian patient to reduce or preclude an allergic reaction in that patient, comprising:
Identifying the patient as one who may suffer from an allergic reaction or is suffering from an allergic reaction due to exposure to an allergen, and
Infusing an amount of magnesium into said patient IV, over a period of no less than 15 minutes and no more than 90 wherein said magnesium, on the basis of elemental magnesium, is from 50-750 mg magnesium.

2. The method of claim 1, wherein said magnesium is infused into said patient over a period of about 15 to about 60 minutes.

3. The method of claim 1, wherein the duration of said infusion is from about 15 to about 30 minutes.

4. The method of claim 1, wherein said magnesium is infused as an aqueous solution.

5. The method of claim 4, wherein said solution comprises sterile water, physiologic saline solution, (Ns), dextrose in water 5% (D5W) or lactated Ringers solution (LR).

6. The method of claim 1, wherein said magnesium is administered to said patient together with co-acting agent selected from histamine blockers, antacids, B-blockers, immune suppressants, corticosteroids, azathioprine, mast cell stabilizers and anti-emetics.

7. The method of claim 1, wherein said magnesium is administered to said patient in conjunction with supportive administration of at least one agent beneficial to said patient which may include at least one vitamin, mineral, trace element, botanical supplement, homeotoxicological preparation, phosphatidylcholine, glutathione, and toxin binders.

8. A method of reducing or preventing allergic reaction to dextran sulfate in a mammalian patient comprising administering a histamine blocker and an antacid to said patient at a time no later than the administration of magnesium into said patient IV, over a period of no less than 15 minutes and no more than 90 minutes in an amount of from 50-750 mg magnesium calculated as elemental magnesium.

9. The method of claim 8, wherein said reaction to dextran sulfate occurs as a result of apheresis in connection with treatment of hyperlipidemia.

10. A method of preventing allergic reaction in a mammalian patient about to undergo a procedure that may trigger an allergic reaction, comprising infusing an amount of magnesium into said patient IV, over a period of no less than 15 minutes and no more than 90 wherein said magnesium, on the basis of elemental magnesium, is from 50-750 mg magnesium, prior to said patient undergoing said procedure.

11. The method of claim 10 wherein said procedure is selected from the group consisting of blood transfusions, apheresis, blood product infusions, plasma exchange, dialysis, treatment with taxane, platinum chemotherapy treatment, immunotherapy, treatment with herceptin, tyrosine kinase inhibitor treatment, PD-1 and PDL-1 inhibitor therapy, and tumor infiltrating lymphocyte treatment.

12. A composition of matter comprising an IV bag containing a preparation of magnesium in an aqueous solution, wherein said preparation comprises 50-500 mg of magnesium measured as elemental magnesium, and wherein said bag is prepared so as to deliver said preparation to a mammalian patient over a period of time of 15-60 minutes- and wherein said IV bag reflects written instructions to deliver the magnesium solution to the patient so as to suppress or reduce an allergic reaction.

13. The composition of claim 12, wherein said aqueous solution comprises sterile water, NS, LR or D5W.

14. The composition of claim 12, wherein said written instructions include an instruction to administer said composition to said patient in advance of a procedure selected from the group consisting of a blood transfusion, plasma exchange, dialysis or apheresis.

15. The composition of claim 12, wherein said aqueous solution comprises at least one one agent beneficial to said patient which may include at least one vitamin, mineral, trace element, botanical supplement, homeotoxicological preparation, phosphatidylcholine and toxin binders.
